(19) United States
(12) Patent Application Publication
(10) Pub. No.: US 2006/0064066 A1
(43) Pub. Date: Mar. 23, 2006

(54) Kind of hand/foot film cover

(76) Inventor: Daniel Wang, Kaohsiung (TW)

Correspondence Address: TROXELL LAW OFFICE PLLC, SUITE 1404, 5205 LEESBURG PIKE, FALLS CHURCH, VA, 22041, US

(21) Appl. No.: 10/942,791

(22) Filed: Sep. 17, 2004

(51) Int. Cl. A61M 35/00

(52) U.S. Cl. 604/292

(57) ABSTRACT

This invention relates to a kind of hand/foot film cover, which consists of an inner layer of cotton-made hand/foot lining and hand/foot back, duly moistened with skin-care solution and an outer layer made of insulating latex having an opening with a fastening structure, the outer layer is on the outside of the inner layer and insulated from the inner layer, making the film cover constructed by the inner and the outer layers available for hands/feet and allowing the outer layer insulate the opening of the inner layer with the fastening structure and forcing the temperature of the skin in contact with the inner layer go up with insulation and open the capillary tubes of the hands/feet while accelerating metabolism and intensifying the absorption of the skin-care solution contained in the inner layer, making the invention an effective means of preserving hands/feet as essential elements in day-to-day works.

### BACKGROUND OF THE INVENTION

The present invention is related to a cover, and more particularly to one kind of hand/foot film cover.

As human skin tends to turn dehydrated in winter when the atmosphere dries up, the hands and feet that are in constant touch with all kind of objects or as one needs to be regular activities or movement. As the skin of the hands and feet is vulnerable to dehydration, with cracks or even bleeding, people tend to buy greasy lotions or skin-care items to fight these phenomena. As one always has the hands and the feet in constant movement, the time-consuming care is usually conducted when one has time or at rest. Caring while working would be almost impossible, as when one wears gloves, the lotion would be erased when it is in touch with the glove, something that would only worsen the care.

Further, a conventional care, which deals with direct application of the item on the hands and the feet, where the lotion is expected to penetrate into the skin, is not as efficient, because it would depend on place and time and, without having the capillary tubes opened in the first place, the skin-care lotion applied on the skin would only remain on top of the skin, creating almost no effect at all.

### SUMMARY OF THE INVENTION

The main purpose of this invention is to provide a film cover construction for efficient care of the hands and feet without the shortcomings of conventional care while allowing preserve the hands and feet as essential elements in day-to-day works.

The other purpose of this invention is to provide a film cover construction that makes the skin temperature go up while accelerating metabolism and intensifying the absorption of the skin-care solution.

To achieve the purposes, this invention contains an inner layer and an outer layer, wherein:
The inner layer comes with absorptive non-woven or cotton-made soft hand and foot lining and hand and foot back, and the inner layer is dipped into the lotion bag to adsorb the moistening lotion to form a hand/foot film;
The outer layer, which is a cover made of soft and insulating plastic or rubber and matches with the shape of the hands/feet and is insulating to the skin lotion and at the opening of the outer layer, there is a fastening structure. To use it, take the outer layer that is moistened with skin lotion from the bag and place it on the outer side of the inner layer, making the inner layer be wrapped inside the outer layer and then make the fastening structure at the opening of the outer layer fasten to close and isolate the opening, thus making the temperature of the hands/feet wrapped inside the inner layer go up to accelerate metabolism and force the capillary tubes to open, allowing the hands/feet adsorb the lotion inside the inner layer in a successful skin-moistening process.

The present invention can be best understood through the following description and accompanying drawings wherein:

### BRIEF DESCRIPTION OF THE DRAWINGS

FIG. 1 is a perspective exploded view of a preferred embodiment of the present invention;

FIG. 2 is an assembled perspective view according to FIG. 1;

FIG. 3 is a diagram of the lotion bag of the present invention;

FIG. 4 is another embodiment of the fastening structure of the present invention;

FIG. 5 is another embodiment of the fastening structure of the present invention;

FIG. 6 is another embodiment of the fastening structure of the present invention;

FIG. 7 is a perspective exploded view of another preferred embodiment of the present invention; and

FIG. 8 is an assembled perspective view according to FIG. 7.

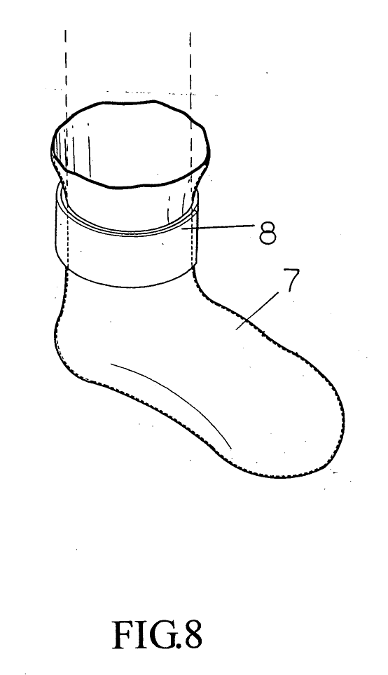

### DETAILED DESCRIPTION OF THE PREFERRED EMBODIMENTS

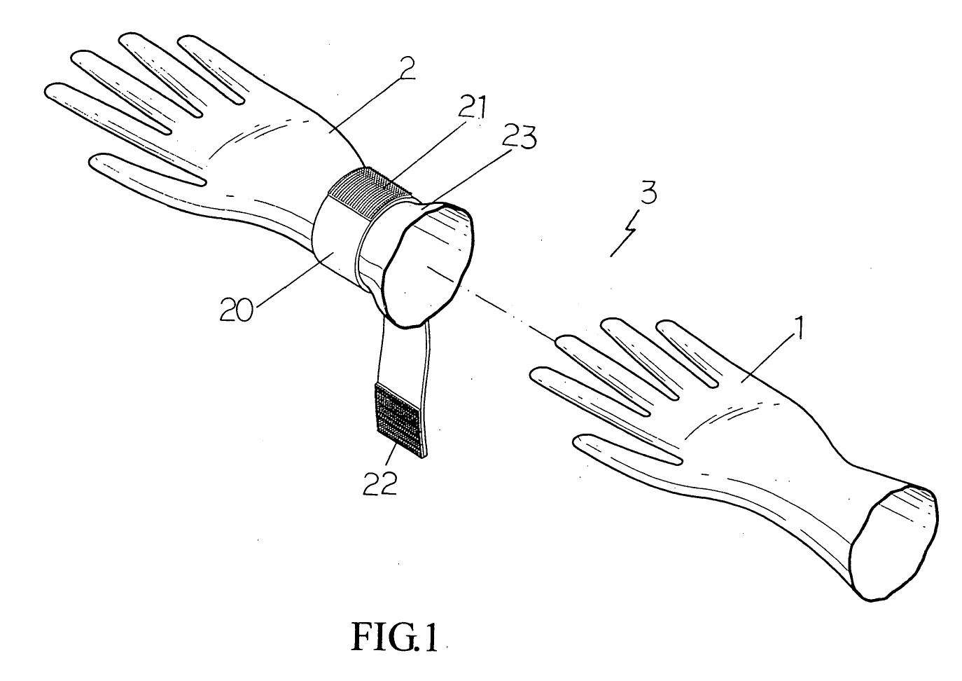

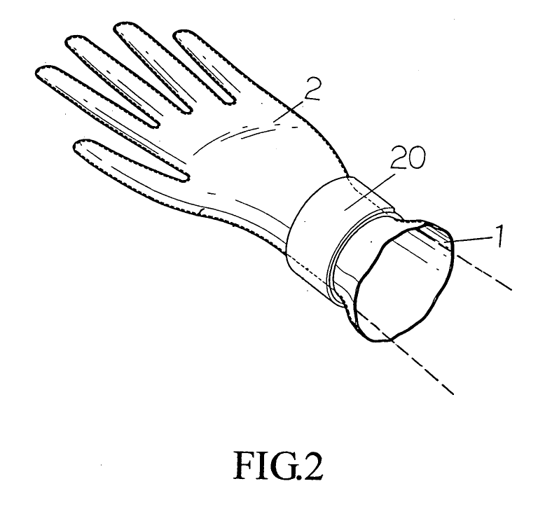

As shown in FIG. 1 and FIG. 2, the hand/foot film cover 3 of this invention consists of a outer layer 2, a inner layer 1 and a fastening structure 20 located at the opening of the wrist 23, which when forced to close by the fastening structure 20, the inner layer 1 is wrapped inside the outer layer 2, forming thus a double-layered film cover 3.

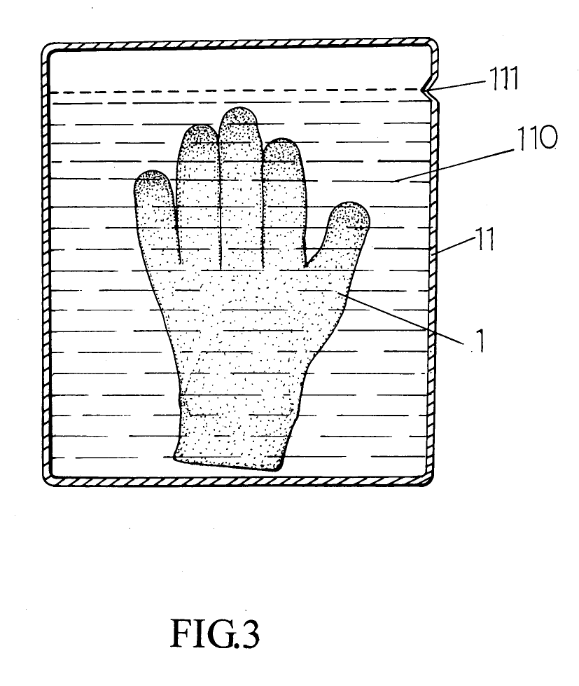

The inner layer 1 has its hand/foot lining and back made of soft yet adsorptive cotton fabric, which is to be placed in a lotion bag 11 to equably absorb the moistening solution 110 in the lotion bag 11, the lotion bag 11 comes with an opening 111. The moistening solution 110 contained is made of levogyration C in 0.5%, collagen in 4%, hyalurnic acid in 6%, oleum rosae in 10%, liquilitigenin in 3%, pectine in 1%, aloe in 2%, citric acid in 0.45%, essence in 0.05%, ion solution in 7% and other elements. (See FIG. 3)

The outer layer 2 is a layer in separate from the inner layer 1 and it has a fastening band 20 at the opening of the wrist, the fastening band 20 comes with one end secured onto the outer layer 2 and a veltro strip 21 is provided. The other end of the band comes with another veltro strip 22. To make use of this film cover on the hands, first tear open the lotion bag 11 from the opening 111 and remove the inner layer 1 that has been moistened with the lotion 110 and wear the inner layer on the hand first, then wear the outer layer 2 on the outside of the inner layer 1, wrapping the inner layer 1 inside the outer layer 2 as one and then, with the fastening band 20 on the outer layer 2, wrap the veltro strip 22 at the end of the band around the opening 23 of the inner layer 1 and against the veltro strip 21 on the band, forcing thus the inner layer 1 to close and by separate from the outer layer 2.

As the inner layer 1 is closed and isolated from the outer layer, skin temperature of the hand in touch with the inner layer build up, forcing the capillary tube of the skin to open for rapid absorption of the lotion contained in the inner layer 1, in a successful skin care by accelerated metabolism and intensified absorption of nutrients.

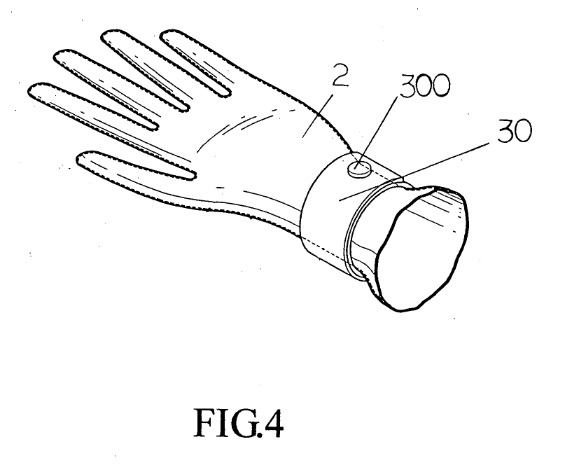

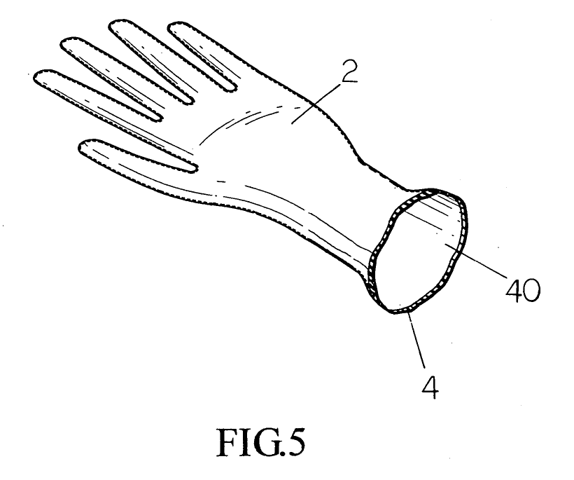

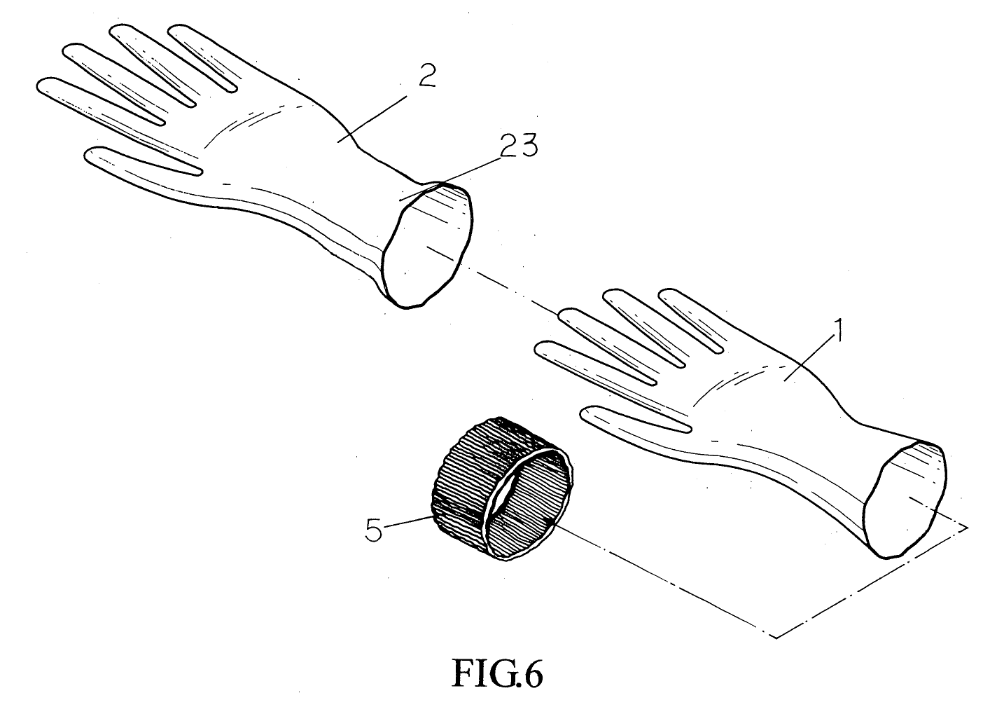

FIG. 4 shows this invention with the outer layer 2, the fastening structure is a band 30 with latches 300 (such as buckles) instead of veltro strips, allowing the fastening band be fastened and the inner layer 1 be closed. FIGS. 5 and 6 show other embodiments of the fastening structure at the opening of the wrist of the outer layer 2 of this invention. In FIG. 5, the fastening structure is an elastic band 4 passing through the opening of the wrist 23 to secure, the opening 23 is then closed and off. The fastening structure can be an elastic loop 5 in FIG. 6, which is available for fasten the opening 23 too to keep the outer layer 2 insulated from the inner layer 1.

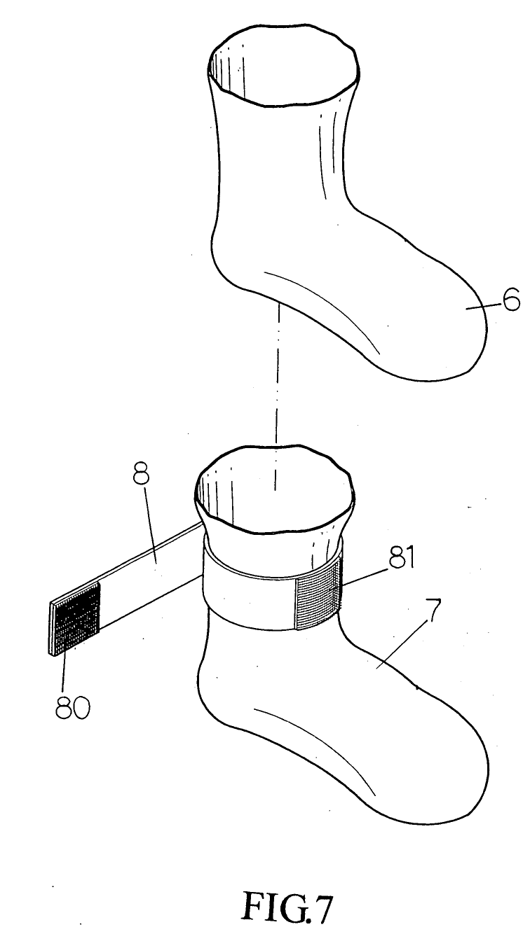

FIG. 7, 8 show another embodiment of this invention when it comes to foot care. Likewise, it is consisted of the inner layer 6 and the outer layer 7. The inner layer 6 is moistened with the skin-care lotion and the outer layer 7 is made of impermeable materials and the opening of the outer layer 7 comes with a fastening structure 8 with a veltro strip 80 at the end of the band that latches the veltro strip 81 to close and insulate the inner layer 6.

What is claimed is:

1. A kind of hand/foot film cover, including an inner layer and an outer layer, wherein the inner layer being made of a kind of soft yet adsorptive cotton in the form that matches the hand/foot, evenly moistened with skin-care lotion as a film for the hand/foot;
the outer layer being a kind of cover made of soft yet insulating latex (silicone) in the form of hand/foot, with a fastening structure at the opening of the wrist; whereby wear the inner layer on the hand/foot and then the outer layer on the inner layer, making the inner layer be wrapped on the inside of the outer layer and then make the fastening structure close the opening of the inner layer forcing it to close and isolate; the skin temperature of the hand/foot inside the layer goes up and the capillary tubes open, the skin will rapidly absorb the lotion contained inside the inner layer, making the film cover provide care to the hand/foot while allowing the hand/foot carry out day-to-day works.

2. The hand/foot film cover as claimed in claim 1, wherein the fastening structure is a fastening band with both the tail end and the upper end attached with a veltro strip.

3. The hand/foot film cover as claimed in claim 1, wherein the fastening structure is a band with latches.

4. The hand/foot film cover as claimed in claim 1, wherein the fastening structure is an elastic loop.

5. The hand/foot film cover as claimed in claim 1, wherein the fastening structure is an elastic band passing through the opening end of the outer layer.
